(19) United States
(12) Patent Application Publication
(10) Pub. No.: US 2025/0186013 A1
(43) Pub. Date: Jun. 12, 2025

(54) Tomosynthesis Imaging System Including a Guidance System With X-Ray Tomosynthesis Registration and Tracking

(71) Applicant: nView Medical Inc., Salt Lake City, UT (US)

(72) Inventors: Cristian ATRIA, Salt Lake City, UT (US); Kevin T. FOLEY, Germantown, TN (US); Mark ELLIOTT, Salt Lake City, UT (US)

(21) Appl. No.: 18/953,623

(22) Filed: Nov. 20, 2024

Related U.S. Application Data
(60) Provisional application No. 63/601,120, filed on Nov. 20, 2023.

(51) Int. Cl. A61B 6/00; A61B 6/03

(52) U.S. Cl. CPC A61B 6/5235; A61B 6/035; A61B 6/485; A61B 6/547

(57) ABSTRACT

An imaging system can include a cone beam tomosynthesis imaging system configured to create a 3D image of a patient anatomy upon an initiation input. The system can also include at least one memory device that has instructions that, when executed by at least one processor, cause the tomosynthesis imaging system to execute multiple actions including obtain the 3D image from the tomosynthesis imaging system; obtain a prior 3D image of the patient anatomy; and register the 3D image with the prior 3D image to produce a composite 3D image. The navigation system can further include an interface system which accepts the initiation input to obtain the 3D image and renders at least a portion of the composite 3D image for review by a supervisor.

### RELATED APPLICATION

This application claims priority to U.S. Provisional Application No. 63/601,120, filed Nov. 20, 2023, which is incorporated herein by reference.

### BACKGROUND

Registration methods in surgery allow a 3D image, such as CT, CBCT, or MRI 3D images, to be registered to a tracking system, such as an optical tracking system or a surgical robotic system. This allows the tracking system, practitioner, and/or robotic system to accurately navigate. Various registration methods exist; however, these approaches tend to exhibit deficiencies such as being computationally expensive, incur excessive x-ray exposure, and lack precision. One registration method is paired point registration. In this method, points are selected on the image and then touched with the tracking system. The registration is achieved by finding a transformation that best aligns points in an image space with points in the tracking space. Another registration method is surface mapping where a point-cloud set of points is collected by sliding a tracked instrument over a surface, such as skin or bone. The surface is found in the image via a process such as segmentations with the registration being achieved by finding the transformation that best aligns the image surface with the point-cloud dataset.

Further registration methods include optical based surface mapping, 2D/3D registration, and automatic registration. Optical based surface mapping is similar to surface mapping, but instead of collecting a point-cloud set of points to obtain the surface in tracking space, a 3D optical system collects cloud-points of the surface in tracking space. In 2D/3D registration, a discrete number of 2D images, such as from fluoroscopic projections, are used to register a 3D image. In this case, a fluoroscopic C-arm can be tracked so that a projection geometry of the 2D images is known in tracking space, with the registration being achieved by finding the transformation that best aligns a virtual projection of the 3D image with the fluoroscopic projection. In automatic registration, an intraoperative 3D imaging system is tracked in camera space, where the position of the image is known with respect to the position of the imaging system. Therefore, the position of the image is known in tracking space.

However, each of these registration methods has certain drawbacks. For example, paired-point registration is a manual process that can add to the surgical time, is error prone, and when too few points are collected, it can lead to registration errors. Further, this method cannot be practically performed inside the body, and therefore cannot be used in minimally invasive surgery near the area of interest. Surface mapping can be inaccurate if the surface is not rigid (i.e. dependent on applied pressure), or if the surface segmented from the 3D image does not match the surface that is touched. This method also cannot be practically performed inside the body, and therefore cannot be used in minimally invasive surgery near the area of interest.

Similarly, optical based surface mapping requires the bone to be well cleaned, which can be time consuming. As with other approaches, optical surface mapping cannot be practically performed inside the body, and therefore cannot be used in minimally invasive surgery near the area of interest. Further, 2D/3D registration requires the motion of the C-arm in multiple discrete positions to make the registration accurate, while also requiring some ionizing radiation to register. Finally, although automatic registration does not require manual steps, the quality of the intraoperative image may not be as good as the quality from a pre-operative CT. Although registration is fast, image acquisition and reconstruction is not, while also requiring a significant ionizing radiation. It is also important to note that all of the methods previously discussed that register a prior 3D image, such as a CT image, suffer from the fact that even if the registration method was perfect, the anatomy of the patient may not match the image being registered at the time of tracking. For these and other reasons, improvements in imaging and registration during patient procedures continue to be sought.

### SUMMARY

An imaging system can include a cone beam tomosynthesis imaging system that is configured to create a 3D image of a patient anatomy upon an initiation input. The imaging system can also include at least one memory device that has instructions that, when executed by at least one processor, cause the tomosynthesis imaging system to execute multiple actions including obtain the 3D image from the tomosynthesis imaging system, obtain a prior 3D image of the patient anatomy, where the prior 3D image is at least one of a higher quality produced by the cone beam tomosynthesis imaging system, or is produced via a different imaging system than the 3D image. The instructions can also cause the system to register the 3D image with the prior 3D image to produce a composite 3D image. The navigation system can further include an interface system configured to accept the initiation input to obtain the 3D image and to render at least a portion of the composite 3D image for review by a supervisor.

Another example of the present disclosure is a method of surgical navigation using a tracked patient reference. The method can include obtaining a 3D image from a tomosynthesis imaging system and obtaining a prior 3D image of the patient anatomy where the prior 3D image is at least one of higher quality produced by the cone beam tomosynthesis imaging system, or is produced via a different imaging system than the 3D image. The method can also include registering the 3D image with the prior 3D image using the tracked patient reference to produce a composite 3D image. Additionally, the method can further include rendering at least a portion of the composite 3D image to a supervisor.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a schematic of an imaging system in accordance with an example of the present technology including a patient bed to support a patient to be imaged.

FIG. 2A is a schematic of a hybrid imaging system in accordance with an example of the present technology.

FIG. 2B is a schematic showing the hybrid imaging system in a maximum upper position in accordance with one example of FIG. 2A.

FIG. 2C is a schematic showing the hybrid imaging system in a maximum lower position in accordance with one example of FIG. 2A.

FIG. 3 is a perspective view of a stereotactic camera and a robotic arm including tracking markers in accordance with an example of the present technology.

FIG. 4 is a flowchart illustrating an example method of surgical navigation of a patient in accordance with examples of the present disclosure.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “an x-ray source” includes reference to one or more of such devices and reference to “obtaining” refers to one or more of such actions.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, or combinations of each.

As used herein, the term, “3D image” refers to a volumetric three-dimensional image created by a 3D imaging system. Such images can be displayed or used in whole or in part. For example, slices of a 3D image can be displayed rather than an entire 3D image.

As used herein, “degrees of freedom” refers to independent orientations and locations of various objects relative to other objects that can be tracked using the tracking systems and reference markers described herein. For example, the degrees of freedom can include six degrees of freedom for moving a rigid object in space, which include positions along three axes and rotational motion about three axes. Additional degrees of freedom beyond these six can include motions such as flexing and twisting motions between bodies of rigid tissue connected by flexible tissue.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein.

Accordingly, the scope of the invention should be determined solely by the appended claims and their legal equivalents, rather than by the descriptions and examples given herein.

### Example Embodiments

Visual images of patient anatomy can be obtained by scanning procedures such as X-ray, computed tomography (CT) scan, cone beam CT (CBCT), and magnetic resonance imaging (MRI). These methods can be used before a surgery to take an image of the surgical site, and may also be used periodically during surgery. However, these methods cannot be used for continuous monitoring of the patient anatomy and tend to not be practical to continuously repeat the scanning process throughout a surgery. Therefore, scanned images can be combined with surgical navigation systems to provide visualization of surgical instrumentation or implants. Such devices can be displayed as an overlay over an image of the patient anatomy for the surgeon to view, or as an input for robotic or intelligent surgical systems to perform, for example, surgical planning or surgical analysis. Such an overlay can be accurately localized and represented with respect to the image via a registration process. Because the patient anatomy can move with respect to the tracking camera, the anatomy can be tracked to maintain proper registration between surgical devices, displayed images, and actual anatomy.

The imaging and registration systems presented herein can be used to allow a 3D image to be registered to a tracking system and periodically updated throughout a patient procedure. In particular, the 3D image can be registered to an optical tracking system or a surgical robotic system. This allows either the tracking system or surgical robotic system to accurately navigate. In other words, this allows the tracking system to display the location of a surgical instrument as an overlay on the 3D image.

The present disclosure allows for a prior 3D image and a tomosynthesis 3D image to be jointly displayed in an overlay once the image is registered. This allows for objects to be shown that are present intraoperatively in the image, or that were not present in the prior image, making the navigated image more accurate and representative of current conditions. These objects can then be used to exclude certain regions to ensure that the registration is only performed on anatomical information and not on foreign objects that may reduce registration accuracy.

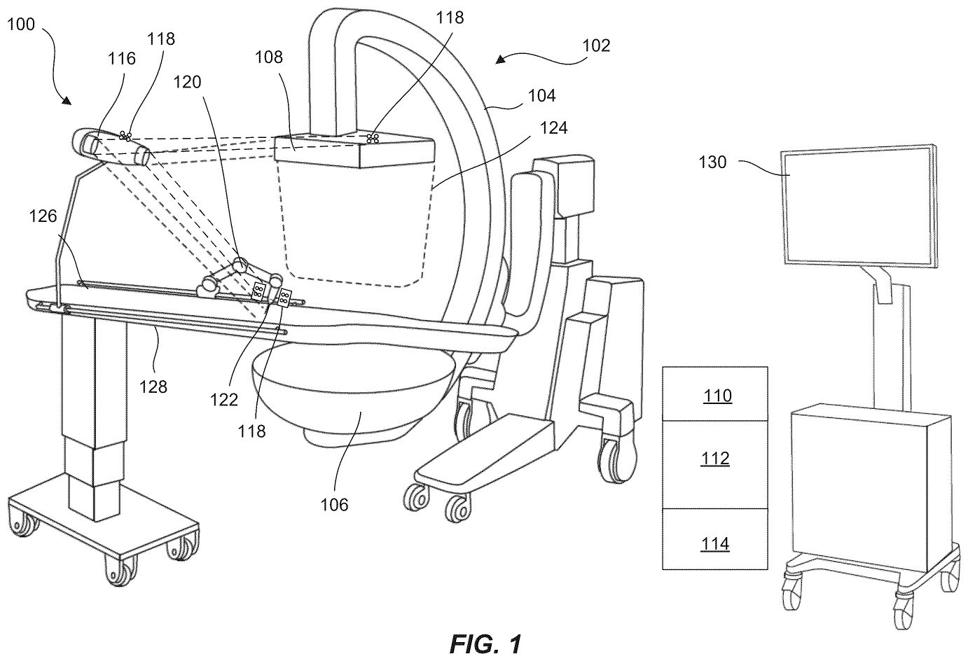

Turning now to FIG. 1, a navigation system 100 is described. During surgery, the imaging and navigation system 100 can be used to display a surgical instrument 122 or instruments as an overlay on a 3D image. This can assist a surgeon or robotic system performing the surgery. The imaging and navigation system 100 can include an imaging system 102. The imaging system 102 is a cone beam tomosynthesis imaging system, i.e. a tomosynthesis system capable of producing tomographic images while imaging with an X-ray source remaining on one side of one plane where imaged object lays (the X-ray source hemisphere), and an X-ray detector remaining substantially opposite to the X-ray source (the X-ray detector hemisphere) during the data acquisition process, with both hemispheres being disjointed and non-overlapping. Exemplary cone beam tomosynthesis imaging systems are described in U.S. Pat. Nos. 10,070,828; 10,846,860; and 12,062,177 which are incorporated herein by reference. One example of an imaging system is a tomosynthesis is imaging system that is a hybrid system. In addition, if providing tomosynthesis images, the system can provide one or both of fluoroscopy or CBCT imaging. Cone beam tomosynthesis can utilize proprietary reconstruction and visualization technologies to create near real-time 3D images via rapid reconstructions of continuously detected 2D projections. In some examples, the cone beam tomosynthesis is cone beam tomosynthesis fluoroscopy (CBTF), or cone beam computed tomography (CBCT). CBTF is an imaging technique that provides real-real time images of a target anatomy. However, CBTF provides images with no depth information, or in other words, in 2D. CBCT is a fully three-dimensional imaging technique, which allows reconstructions with isotropic voxels and high spatial resolution, allowing for the precise measurement or imaging of a selected anatomy in all three orthogonal planes. The imaging system 102 can be a 3D C-arm device 104, including an x-ray source assembly 106 attached to the lower portion of the C-arm device 104 and a detector array 108 attached to the upper portion of the C-arm device 104.

The imaging system 102 can be oriented so as to collect a 3D image from a target image formation region 124. This image formation region 124 can be directed toward a patient laying on a support table 126. For coarse adjustments, one or both of the support table 126 and the imaging system 102 can be moved relative to one another. This may be accomplished by manually moving one or both the support table and the imaging system (e.g. via wheels). For finer adjustments to orientation and location of the image formation region, the imaging system itself can be adjusted. For example, a C-arm support frame can be rotated, x-ray sources can be tilted, and/or detectors can be adjusted. The support table 126 can optionally include side rails 128 which can be used as mounting locations for additional devices, as a handle for moving the support table, or as a safety guide to prevent patient injury.

In one example, the imaging system 102 can create a 3D image of a patient anatomy. The patient anatomy can be in any anatomical region such as, but not limited to, spinal column, hip joints, knee joints, hands, wrists, feet, ankles, shoulders, bone fragments, skull, etc. In some examples, the imaging system 102 can create the 3D image either upon an initiation input from a user or automatically by a coded decision maker. The initiation input can be the push of a button, stepping on a foot pedal, voice command, or the like. Additionally, in the examples where creating the 3D image is automatically generated by a coded decision maker, the initiation input can be generated via a pre-set list of instructions on when to create the 3D image, a timer system where the 3D image is created at a predetermined time, by using artificial intelligence (e.g. dynamically as needed based on experience and input guidelines), or the like. As for the actual image created, in some cases, the imaging system 102 provides volumetric imaging data, i.e. reconstructs a 3D volume and provides volumetric data. This volumetric data can be rendered as various views on a display for a human user, or can be directly used by the algorithm or artificial intelligence without producing a view on the display.

The imaging and navigation system 100 can further include at least one memory device 110 and at least one processor 112. The memory device 110 can have instructions that, when executed by the processor, cause the imaging system 102 to execute multiple actions. The imaging system and navigation system 100 can be instructed to obtain the 3D image of the patient anatomy from the imaging system 102. The 3D image can be an updated or current image which may reflect changes in position of tissue and/or surgical tools. In some examples, the 3D image is obtained using a short-angle acquisition. Such short-angle acquisitions can involve rotating an x-ray source along a limited arc such that limited data is collected. This limited arc can be varied depending on a desired degree of resolution or acquisition time. However, as a general guideline, the limited arc can range from 5° to less than 360°, in some cases 10° to 45°, and in other cases 15° to 30°. Similarly, the x-ray source can be passed along this limited arc a single time per acquisition, or multiple times per image acquisition. Regardless, this can result in images which are of lower quality but which may nonetheless provide coarse updates to changes in the image formation region 124.

The imaging and navigation system 100 can also be instructed to obtain a prior 3D image of the patient anatomy. The prior 3D image can be of higher quality produced by the imaging system 102 (e.g. a CBCT image, or a higher resolution or higher power image) than the 3D image or can be produced via a different imaging system than the 3D image. For example, a patient can be imaged during an earlier visit (e.g. same day or a previous day) using a high-resolution imaging modality. Examples of where the prior 3D image is of higher quality or has different characteristics than the 3D image can include, but is not limited to, CT, CBCT, or MRI images. In some examples, the prior 3D image can be obtained from a prior CT imaging session. In other examples, the prior 3D image can be the composite 3D image from an earlier iteration using the same imaging system used to produce the 3D image.

The imaging and navigation system 100 can also be instructed to register the 3D image from the imaging system 102 with the prior 3D image. This produces a composite 3D image which represents a combination of features from the prior 3D image and the 3D image (i.e. newly acquired). In some cases, the composite 3D image can be an overlay of the 3D image into the prior 3D image. In this case, the image can include highlights of features that differ between the two images, such as the presence of surgical instruments 122 or implants in the patient, changes in tissue position, etc. In other cases, the composite 3D image can be an integrated image of the 3D image and the prior 3D image. The integrated composite 3D image can be produced by morphing the prior 3D image onto the 3D image to match an updated anatomical condition of the patient. Morphing of the prior 3D image onto the 3D image can include isotropic scale, anisotropic scale, shape morphing, spline transformations, or the like. Additionally, because registering the 3D image with the prior 3D image uses a very rich intraoperative dataset, the morphing can use at least six degrees of freedom to improve the matching of the prior 3D image to the anatomical position of the patient during surgery. In this case, the integrated composite 3D image retains the quality of the prior 3D image while revising the image to reflect new elements and changed features to reflect current conditions.

In one example, the 3D image can be reconstructed in an iterative reconstruction scheme by using the prior 3D image, or a morphed version of it, as a seed to an iterative reconstruction algorithm to produce an integrated image. This can reduce certain artifacts, such as tomosynthesis artifacts. In some instances, the prior 3D image seed is first registered in order for the seed to be placed in a correct space for reconstruction. For example, such registration can be achieved by storing a position of the imaging system at the time a first image is taken. When taking an updated or later tomosynthesis image, the first image can be used as a seed to the tomosynthesis reconstruction algorithm by virtually positioning the seed in the new reference frame of the imaging system given its new position. These positions can be relative to the patient if the patient is being tracked, or with respect to the table. One exemplary reconstruction technique is outlined in U.S. Pat. No. 11,610,346, which is incorporated herein by reference although any suitable image reconstruction technique may be used such as, but not limited to, filtered back projections or similar tomographic techniques.

In some examples, the imaging and navigation system 100 can further include an interface system 114. The interface system 114 can be configured to accept an initiation input, such as an initiation input from a user that causes the imaging system 102 to create a 3D image of the patient anatomy. The interface system 114 can be any suitable input device. Non-limiting examples of suitable input devices can include keyboard, touchscreen, manual button, or in the case of an automated supervisor the input device can be the processor 112 itself. Additionally, the interface system 114 can further be configured to obtain the 3D image from the imaging system 102 and to render at least a portion of the composite 3D image for review by a supervisor, thus allowing for intraoperative decision making. In some instances, the supervisor can be a human user, such as a surgeon, physician's assistant, nurse, etc. In other instances, the supervisor can be a coded decision maker, such as an automated computer system, a pre-programed decision model, artificial intelligence, etc., or a combination thereof. In some examples, the interface system 114 can be a display 130 onto which the composite 3D image is rendered for the user. In other examples, the interface system 114 can render the composite 3D image directly to the coded decision maker. In yet another example, the interface system 114 can provide information that describes the modifications of the prior image to the coded decision maker without explicitly providing the composite image. The interface system (e.g. including the processor, memory device, display, etc) can be connected to the imaging system and the camera via a connection. The connection can be wired or wireless (e.g. Bluetooth, Wi-fi Zigbee, etc).

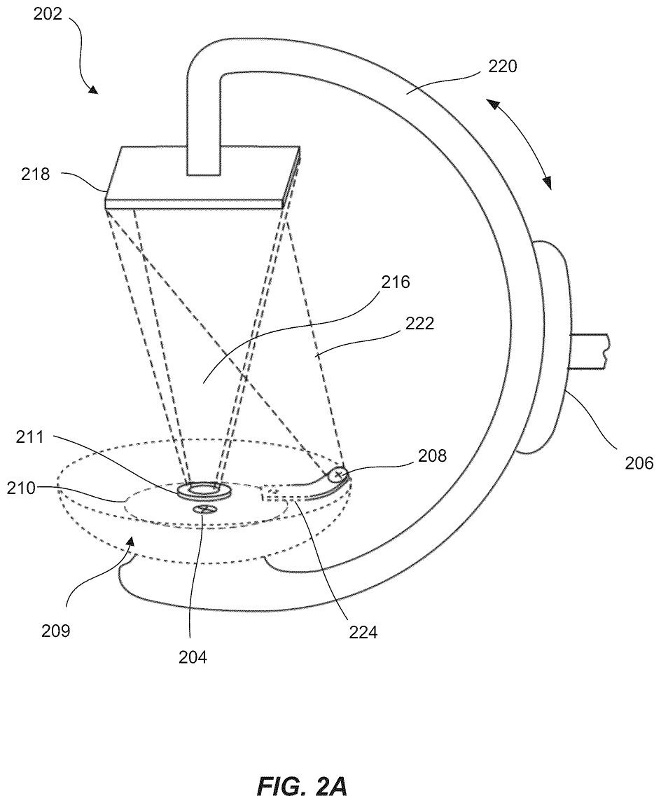

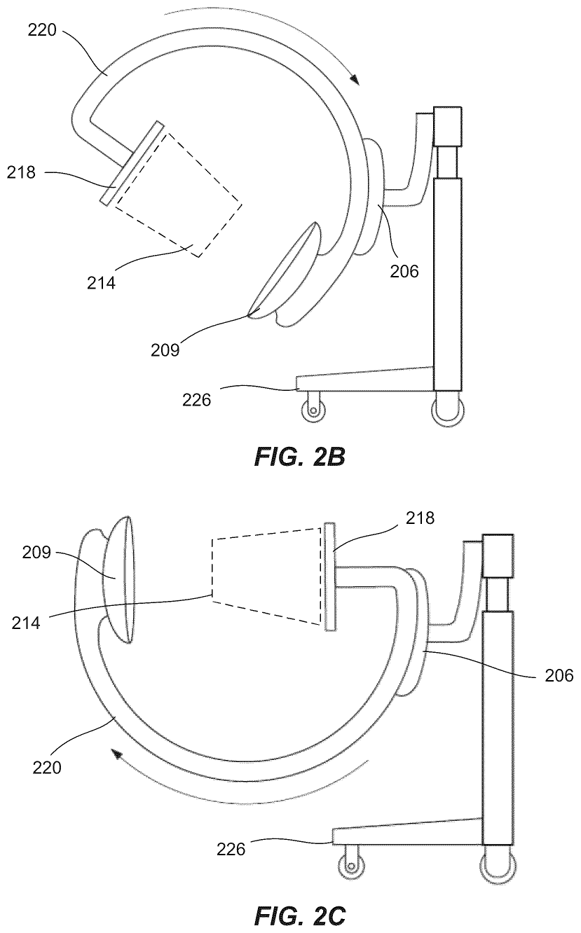

One example of an imaging system is a tomosynthesis is imaging system that is a hybrid system. In addition, if providing tomosynthesis images, the system can provide one or both of fluoroscopy or CBCT imaging. Hybrid systems can include a single x-ray source or multiple x-ray sources. In one example, a common x-ray source is used to generate the cone beam tomosynthesis image and the fluoroscopic images. In this manner, the common x-ray source is configured to be fixed in a parked position during fluoroscopic imaging. Accordingly, a single x-ray source can be used. In another optional example, the hybrid system can include at least one rotatable tomosynthesis x-ray source and at least one central x-ray source. In this case, the at least one central x-ray source can be used to produce the fluoroscopic images while the at least one rotatable x-ray source can produce the tomosynthesis images. FIG. 2A is an example of such a hybrid imaging system 202 having a first stationary x-ray source 204 and a second rotating x-ray source 208 as part of an x-ray assembly 209. The hybrid system can include at least one tomosynthesis x-ray source and at least one central beam x-ray source. The term stationary is intended to specify that the x-ray source is stationary with respect to the imaging system 202 and the image formation region 214 during imaging to produce fluoroscopic images. Thus, a stationary x-ray source can be permanently fixed in a specific location, or temporarily locked into a position during imaging. In some examples, the first x-ray source 204 can be stationary, being attached to the lower portion of the C-arm frame 220. This x-ray source produces a first stationary x-ram beam 216 which is directed toward a detector array 218. Generally, the detector array 218 can be fixed to an upper portion of the C-arm frame 220. The second x-ray source 208 can be secured within a hollow rotary table or bracelet 210 having a continuous loop track, allowing for the second x-ray source 208 to be rotated along the loop track. The rotating second x-ray source 208 enables images to be taken of the patient anatomy from various angles. In one example, the hollow rotary table 210 can surround or encompass the stationary first x-ray source 204, with the stationary first x-ray source 204 being in the hollow portion of the hollow rotary table 210. An optional hollow slip ring 211 can house power transfer and other electronics for controlling the x-ray sources. The second x-ray source 208 produces a rotating x-ray beam 222 (e.g. rotating within a rotation plane) which traverses the image formation region 214 to produce tomosynthesis images. In another alternative, a high voltage power source 224 can be mounted along with the rotating x-ray source 208. The hollow rotary table allows the x-ray beam of the central x-ray source to be unimpeded by the rotating source.

Notably, the C-arm frame 220 can be secured to a base 226 via the C-arm attachment 206. The C-arm attachment 206 can include a coupling mechanism which not only retains the C-arm frame 220 in place but allows the C-arm frame to slide up and down along the frame body. In any of the above configurations, the C-arm frame 220 can slide up and down with respect to the C-arm attachment 206 to allow for orbital rotation to produce CBCT images. This can allow for adjustment of the image formation region 214 relative to the patient (e.g. especially by changing a relative angle) and to facilitate collection of data for CBCT image acquisition. As a further illustration, FIG. 2B shows the C-arm frame 220 in a maximum upper position where the C-arm frame 220 is slid upwards along the attachment 206 to the base 226. At this point, the attachment 206 is oriented adjacent to the x-ray source assembly 209. FIG. 2C shows the C-arm frame 220 slid downward to a maximum lower position where the attachment 206 is aligned adjacent the detector array 218. Of course, the C-arm frame 220 can be slid and oriented at any position along a body of the C-arm frame 220. When creating CBCT images, multiple projections can be taken as the C-arm slides from the limit positions shown in 2B and 2C. In one version, when the imaging system has a central beam source 204 shown in FIG. 2A, the central beam source can be used to produce the projections for CBCT imaging. In another version, the rotating source that is used for tomosynthesis can be substantially parked to produce the CBCT projections as the C-arm slides creating an orbital acquisition as shown in FIG. 2B. In yet another version, two C-arm spins can be combined, where the rotating source is first parked at one of the two intersections between the rotating plane of the source and the orbital plane along which the C-arm slides for its first spin (FIG. 2C position 1), and then the same source is parked at the other intersection point for the second spin (FIG. 2C position 2).

Referring back to FIG. 1, in some examples, the imaging system 100 can further include a tracking camera 116. The tracking camera 116 can be one or both of a visible light camera and an infrared camera. In other examples, multiple cameras or lenses can be used. In further examples, the tracking camera 116 can be a stereoscopic or stereotactic camera. The tracking camera 116 can be a standard NDI navigation camera, an RGB-D camera, or the like. Regardless of the type of tracking camera 116 used, the tracking camera 116 can be configured to track at least one of a surgical instrument 122, the patient, or the imaging system 102. In one example, the tracking camera 116 can track these objects by using reference markers 118, such as light emitting LEDs or via reflector markers positioned at fixed location on at least one of the patient, a surgical instrument, or the imaging system 102. Alternatively, the tracking camera 116 can be used to track these objects via image recognition software (i.e. algorithms, models or the like, including AI based learning models). In this case, the tracking camera 116 can transmit a captured image to a processor where objects are identified (i.e. a tool, specific parts of an imaging device 102, and/or patient anatomy).

In some instances, such as in longer surgeries or when extra precision is desired, an updated 3D image can be acquired to make sure current navigation efforts are accurately guiding procedures to desired tissue. Ideally continuous imaging, or near continuous imaging, would provide maximum precision. However, this is most often not practical due to computational loads, image acquisition times, and can result in undesirable excessive x-ray exposure for the patient. Therefore, in some examples, the imaging system 102 periodically creates a new 3D image of the patient anatomy (e.g. at automated intervals, at dynamically optimized intervals based on modeling, or triggered manually by a clinician). The new 3D image is then re-registered with the prior 3D image or preceding composite 3D image, creating an updated registration. This allows for a surgical instrument 122 or multiple surgical instruments to be tracked based on the updated registration. Thus, the imaging system 102 can create a new 3D image upon an initiation input from a user, at predetermined time intervals, from input from a computer using artificial intelligence, or the like.

In some examples, the imaging system 102 can be calibrated prior to imaging intraoperatively. This allows the image of the patient to be in a known position with respect to the reference markers 118 of the imaging system 102. In other examples, the position of the patient can be found impromptu by having objects of known shape that are visible in the image and that can be tracked by the tracking camera 116 during or immediately before or after imaging intraoperatively. In one example, registering the 3D image with the prior 3D image includes camera-based registration. This includes at least one of a pre-calibrated camera position registration and an object-based registration using an optical image obtained using the tracking camera 116. In some examples, the tracking camera 116 is a stereotactic tracking camera. In another example, registering the 3D image with the prior 3D image includes image-based registration, where common features between the 3D image and the prior 3D image are correlated. Image based registrations can be achieved via iterative methods such as gradient descent or other techniques to solve inverse problems. These problems are often achieved by minimizing a cost function, as such cost function can be different for the different types of imaging being registered. For example, when registering images from MRI to X-ray based tomosynthesis, similarity metrics can be used rather than traditional L-N norms. The registration can be a 3D degree of freedom registration, a rigid registration, or a deformable registration with more degrees of freedom.

In one example, registering the prior 3D image with the 3D image further includes defining a selected local portion of the prior 3D image. In this manner, the prior 3D image is only modified within the selected local portion, producing the composite 3D image using a local matching metric. As a result, computational load is decreased and computing effort is not expended in updating portions of the image which are not important to the procedure.

The local matching metric can be computed dynamically over a region local to a surgical instrument so that the registration is continuously changing and updating as the surgical instrument 122 is moved. In some examples, the local matching metric is L1-norm or L2-norm cost function. However, other matching metrics can be used to align the 3D image with the composite image.

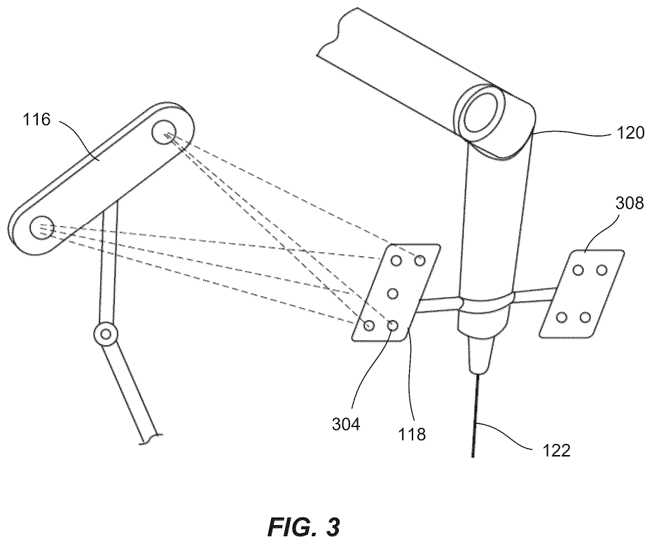

In one example, the navigation system 100 further comprises a robotic arm 120. The robotic arm can be configured to manipulate a surgical instrument 122. It can also include a command input, which can be operatively connected to the supervisor and configured to accept instructions from the supervisor. In some examples, the surgical instrument 122 can include one or more of an implant, a drill, a cutter, forceps, needle drivers, lasers, scissors, clip applicators, cauterizer, hook, or the like. Additionally, the robotic arm 120 can be moved and positioned with respect to the composite 3D image in order to position the surgical instrument 122 where needed. To further explain this process, FIG. 3 shows a close up of the robotic arm 120 and a stereotactic camera 116. The robotic arm 120 can have tracking markers 304 attached, either individually or in the form of an array. In some examples, robotic arm 120 can have a tracking marker array 118 and an image localization array 308. The tracking marker array 118 can be tracked using the tracking camera 116. The image localization array 308 can be positioned to be detected via the detector 108 (FIG. 1) of the imaging system 102. Thus, the robotic arm 120 can move and be positioned, using the tracking camera 116. In this manner, the tracking marker array 118 can be used by visually registering the tool location with respect to the composite 3D image. Thus, either alone or in addition to the tracking marker array 118, the image localization array 308 can be used to align images of the tools within the 3D image and/or 3D composite image. The robot can either execute a predetermined plan based on the prior 3D image, or execute a plan based on the most recent composite image. In yet another version, the predetermined plan based on the prior image can be morphed or modified according to the registration process and be updated to conform to the new disposition of the anatomy as seen in the composite image. In many cases the user would have supervision and control over this new plan and can be allowed to modify it before the robot executes the plan.

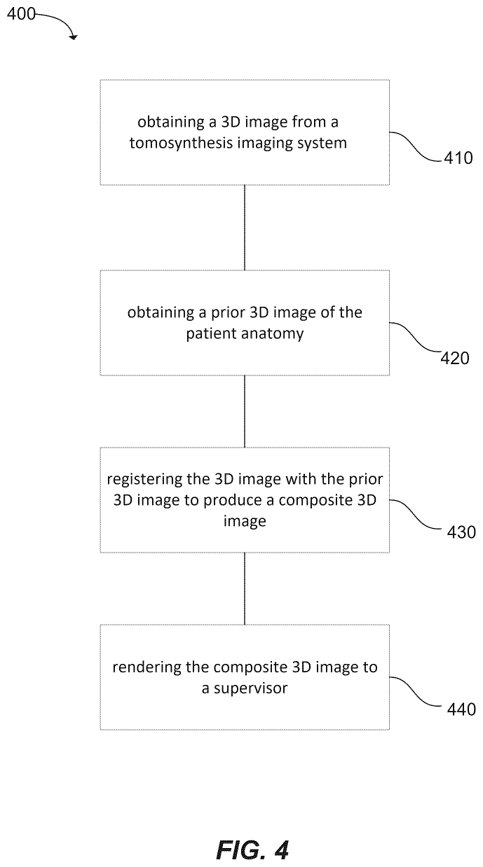

The present disclosure also describes a complementary method of surgical navigation of a patient. FIG. 4 is a flowchart illustrating one example method 400 of surgical navigation of a patient. In some examples, the method 400 can use a patient reference tracking of a patient anatomy. The method 400 can include obtaining a 3D image from a tomosynthesis imaging system 410 and obtaining a prior 3D image of the patient anatomy 420. In some examples, the imaging system can be a tomosynthesis imaging system, a CT imaging system, an MRI imaging system, or any other kind of imaging system that produces a 3D image. In some examples, the 3D image is a prior 3D image, a 3D tomosynthesis image, a computed tomography image, or a combination of these images. In some examples, the prior 3D image can be of higher quality produced by the cone beam tomosynthesis imaging system than the 3D image or can be produced via a different imaging system than the 3D image. Examples of where the prior 3D image is of higher quality is when the tomosynthesis image was first taken at a higher resolution or with additional radiation. Examples of where the prior 3D image has different characteristics than the 3D image is when it is a CT, CBCT, or MRI image. In some examples, the prior 3D image can be obtained from a prior CT imaging session. In other examples, the prior 3D image can be the composite 3D image from an earlier iteration.

In one example, the method 400 can further include registering the 3D image with the prior 3D image 430. Registration can be done by aligning a tracking reference marker at a location that can be identified and/or tracked between each of the 3D image and the prior 3D image to produce a composite 3D image. Such registration can be accomplished using one or both of tracking markers and object recognition. When using tracking markers, these devices can be oriented on one or more of the imaging device, patient, optical camera, and surgical tools as described previously. The tracking reference marker allows for the 3D image and the prior 3D image to be properly aligned, which is needed to produce an accurate composite 3D image. Suitable tracking reference markers can include, but are not limited to one or more light emitting LEDs or reflector markers. Object recognition can also be used alone or in combination with physical tracking markers. For example, object recognition can include image recognition software, AI-driven models, or the like for identifying common features within each image. These common features can then be aligned by registration to a common point or set of points. For example, Hough transforms can be used to identify features and these features can be analyzed for commonality between the two images. Similarly, object detection machine learning algorithms can be used. The reference frame can be relative to that of the initial image or relative to that of the new image, or relative to that of the physical patient.

In one example, the composite 3D image is an overlay of the 3D image onto the prior 3D image. In order to assist a clinician in identifying differences, the overlay composite 3D image can include highlights of features that differ between the two images, such as the presence of surgical instruments, implants in the patient, or shifted tissue. An overlay composite 3D image can be produced by blending certain elements of the new image, by overlaying them, replacing contents of the first image by contents of the second image, or by using functions that combine elements of both images. The composite image can alter the levels of grey of the new image, or add color to show the elements of the new image in the prior image to highlight the differences. In another example, the composite 3D image can be an integrated image of the 3D image and the prior 3D image. This integrated composite 3D image can be produced by morphing the prior 3D image with the 3D image to match an updated anatomical condition of the patient. Morphing can be done either by starting with the prior 3D image and applying morphing algorithms to match the 3D image (i.e. updated image), or by starting with the 3D image and morphing the 3D image using the prior 3D image. Morphing of the prior 3D image with the 3D image can include isotropic scale, anisotropic scale, shape morphing, spline transformations, manifolds or the like. Additionally, because registering the 3D image with the prior 3D image uses a very rich intraoperative dataset, the morphing can use at least six degrees of freedom to improve the matching of the prior 3D image to the anatomical position of the patient during surgery. Morphing can be used to account for brain shifts in cranial neurosurgery procedures or for spine flexing in spine procedures for example. A representation of the morphing itself can also be valuable. In this case, a vector field can be used to display the morphing transformation and show the surgeon how the image has changed. The morphed 3D image can further be morphed during navigation when using non-rigid registration navigation, for example by using distributed patient references such as those described by U.S. Patent Application publication No. US-2023-0310114-A1 which is incorporated herein by reference.

In further examples, the method 400 can include rendering the composite image 440. In some cases, only a portion of the composite image can be rendered. In such cases, the rendered portion can represent a limited subset of the entire composite image. In other cases, the entire composite image can be rendered. In some examples, the composite image can be rendered to a supervisor which is one or both of a human user and a coded decision maker. In some instances, the supervisor can be a human user, such as a surgeon, physician's assistant, nurse, etc. In other instances, the supervisor can be a coded decision maker, such as an automated computer system, a pre-programed decision model, artificial intelligence, etc., or a combination thereof.

In another example, the method 400 can also include tracking the tomosynthesis imaging system to produce the tracking reference location via one or both of tracking markers and object recognition as discussed above. More specifically, the tracking can be done by using a camera. The camera can be one or both of a visible light camera and an infrared camera. In other examples, multiple cameras or lenses can be used. In further examples, the camera can be a stereoscopic or stereotactic camera. The tracking camera can be a standard NDI navigation camera, an RGB-D camera, or the like. In one example, the methods can include stereotactically tracking the tomosynthesis imaging system. In some examples, the tracking can be done by using reference markers. The reference markers can be attached to one or more of the tomosynthesis imaging system, patient, surgical tools, and the like.

While the flowcharts presented for this technology may imply a specific order of execution, the order of execution may differ from what is illustrated. For example, the order of two or more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, a non-transitory machine-readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. An imaging system, comprising;
a cone beam tomosynthesis imaging system includes at least one rotatable tomosynthesis x-ray source and is configured to create a 3D image of a patient anatomy upon an initiation input;
at least one memory device including instructions that, when executed by at least one processor, cause the tomosynthesis imaging system to:
obtain the 3D image from the tomosynthesis imaging system;
obtain a prior 3D image of the patient anatomy, wherein the prior 3D image is at least one of a higher quality produced by the cone beam tomosynthesis imaging system, or is produced via a different imaging system than the 3D image; and
register the 3D image with the prior 3D image to produce a composite 3D image; and
an interface system configured to accept the initiation input to obtain the 3D image and to render at least a portion of the composite 3D image for review by a supervisor.

2. The imaging system of claim 1, wherein the cone beam tomosynthesis imaging system is a hybrid system that is configured to produce either of fluoroscopic images and cone beam computed tomography (CBCT) images.

3. The imaging system of claim 2, wherein a common x-ray source is used to generate the CBCT images or the fluoroscopic images wherein the common x-ray source is configured to be fixed in a parked position during at least one of fluoroscopic imaging or CBCT imaging.

4. The imaging system of claim 2, wherein the hybrid system further includes at least one central x-ray source, wherein the at least one central x-ray source is used to produce one or both of the fluoroscopic images and the CBCT images.

5. The imaging system of claim 1, further comprising positional encoders configured to track a position and orientation of the 3D image relative to the prior 3D image.

6. The imaging system of claim 1, further comprising a stereotactic tracking camera, wherein the stereotactic tracking camera tracks via at least one reference marker positioned at fixed locations on at least one of the patient and the tomosynthesis imaging system.

7. The imaging system of claim 6, wherein at least one of:
a. the stereotactic tracking camera is further configured to track at least one surgical instrument;
b. the at least one reference marker is one or more of light emitting LEDs and reflector markers; and
c. the fixed locations includes both the patient and the tomosynthesis imaging system.

8. The imaging system of claim 7, wherein the cone beam tomosynthesis imaging system periodically creates a new 3D image of the patient anatomy, re-registers the new 3D image with the composite 3D image creating an updated registration, and allowing the at least one surgical instrument to be tracked based on the updated registration.

9. The imaging system of claim 7, wherein the user interface system displays the location of the at least one surgical instrument overlaid over the prior 3D image.

10. The imaging system of claim 7, wherein registering the 3D image with the prior 3D image includes camera-based registration which includes at least one of a pre-calibrated camera position registration and an object-based registration using an optical image obtained using the stereotactic tracking camera.

11. The imaging system of claim 1, wherein registering the 3D image with the prior 3D image includes image-based registration, wherein common features between the 3D image and the prior 3D image are correlated.

12. The imaging system of claim 1, wherein the composite 3D image is an overlay of the 3D image into the prior 3D image, including highlights of features that differ.

13. The imaging system of claim 1, wherein the composite 3D image is an integrated 3D image produced by morphing the prior 3D image onto the 3D image to match an updated anatomical condition of the patient.

14. The imaging system of claim 13, wherein morphing the prior 3D image onto the 3D image comprises isotropic scaling, anisotropic scaling, shape morphing, spline transformations, or a combination thereof.

15. The imaging system of claim 13, wherein registering the 3D image with the prior 3D image uses at least 6 degrees of freedom to morph the prior 3D image onto the 3D image to produce the composite 3D image.

16. The imaging system of claim 1, wherein the prior 3D image is obtained from a prior CT imaging session or is the composite 3D image from an earlier iteration; and the 3D image from the tomosynthesis imaging system is obtained using a short-angle acquisition.

17. The imaging system of claim 1, wherein registering the prior 3D image onto the 3D image further comprises defining a selected local portion of the prior 3D image such that the prior 3D image is only modified within the selected local portion to produce the composite 3D image using a local matching metric.

18. The imaging system of claim 1, wherein the supervisor is a user and the interface system is a display onto which the composite 3D image is rendered for the user.

19. The imaging system of claim 1, wherein the supervisor is a coded decision maker, and the interface system renders the composite 3D image to the coded decision maker.

20. The imaging system of claim 1, wherein the initiation input is configured to be manually generated by a user or automatically generated by a coded decision maker.

21. The imaging system of claim 1, further comprising a robotic arm configured to manipulate a surgical instrument which includes a command input which is operatively connected to the supervisor and configured to accept instructions from the supervisor.

22. A method of surgical navigation of a patient using distributed patient reference tracking of a patient anatomy, comprising:
obtaining a 3D image from a tomosynthesis imaging system;
obtaining a prior 3D image of the patient anatomy, wherein the prior 3D image is at least one of a higher quality produced by the tomosynthesis imaging system, or is produced via a different imaging system than the 3D image;
registering the 3D image with the prior 3D image using a tracking reference location to produce a composite 3D image; and
rendering at least a portion of the composite 3D image to a supervisor.

23. The method of claim 22, wherein the 3D image is a prior 3D image, a 3D tomosynthesis image, a computed tomography image, or a combination thereof.

24. The method of claim 22, further comprising stereotactically tracking the tomosynthesis system to produce a tracking reference location.

25. An imaging system, comprising;
a cone beam tomosynthesis imaging system configured to create a 3D image of a patient anatomy upon an initiation input,
wherein the cone beam tomosynthesis is a hybrid system that is configured to produce either of fluoroscopic images and cone beam computed tomography (CBCT) images;
a stereotactic tracking camera configured to track at least one of the tomosynthesis imaging system and at least one surgical instrument;
at least one memory device including instructions that, when executed by at least one processor, cause the tomosynthesis imaging system to:
obtain the 3D image from the tomosynthesis imaging system;
obtain a prior 3D image of the patient anatomy, wherein the prior 3D image is at least one of a higher quality produced by the cone beam tomosynthesis imaging system, or is produced via a different imaging system than the 3D image; and
register the 3D image with the prior 3D image to produce a composite 3D image, wherein the composite 3D image is produced by morphing the prior 3D image onto the 3D image to match an updated anatomical condition of the patient; and
a user interface system configured to accept the initiation input and obtain the 3D image and to render at least a portion of the composite 3D image for review by a supervisor, wherein registering the 3D image with the prior 3D image uses at least 6 degrees of freedom to morph the prior 3D image onto the 3D image to produce the composite 3D image, wherein registering the prior 3D image onto the 3D image further adapted to define a selected local portion of the prior 3D image such that the prior 3D image is only modified within the selected local portion to produce the composite 3D image using a local matching metric, and wherein at least one tracked surgical instrument is displayed as an overlay to the composite image.
